(19) United States
(12) Patent Application Publication
(10) Pub. No.: US 2024/0271165 A1
(43) Pub. Date: Aug. 15, 2024

(54) METHOD FOR PRODUCTION OF AMMONIA, USING INORGANIC NANOPARTICLE-MICROBIAL COMPLEX

(71) Applicant: KOREA ADVANCED INSTITUTE OF SCIENCE AND TECHNOLOGY, Daejeon (KR)

(72) Inventors: Doh Chang LEE, Daejeon (KR); Sungjun KOH, Daejeon (KR); Yoojin CHOI, Seoul (KR); Ilsong LEE, Daejeon (KR); Gui-Min KIM, Daejeon (KR)

(21) Appl. No.: 18/681,047

(22) PCT Filed: Aug. 12, 2021
(86) PCT No.: PCT/KR2021/010750

§ 371 Date: Feb. 3, 2024

(51) Int. Cl. C12P 3/00; B01J 27/057; B01J 27/14; B01J 31/00; B01J 31/26; B01J 35/39; B01J 35/45; B01J 35/53

(52) U.S. Cl. CPC C12P 3/00; B01J 27/0573; B01J 27/14; B01J 31/003; B01J 31/26; B01J 35/39; B01J 35/45; B01J 35/53

(57) ABSTRACT

The present invention relates to a method for production of ammonia, using an inorganic nanoparticle-microbial complex in which a nitrogen fixation reaction in a microorganism is improved by increasing the amount of inorganic nanoparticles entrapped in the microorganism. The present invention can produce ammonia at low temperature and low pressure conditions, compared to the conventional Haber-Bosch process of producing ammonia in high temperature and high pressure conditions and in a friendly environmental manner without emission of carbon dioxide that is released during conventional chemical synthesis processes, whereby the present invention may be a competitive alternative to the prior art for production of ammonia that has an unlimited potential as a future energy resource.

### TECHNICAL FIELD

The present invention relates to a method for producing ammonia using an inorganic nanoparticle-microbial complex, and more particularly to a method for producing ammonia using an inorganic nanoparticle-microbial complex in which the nitrogen fixation reaction in a microorganism is improved by increasing the amount of inorganic nanoparticles entrapped in the microorganism.

### BACKGROUND ART

Ammonia (NH₃) is used in a variety of industries, including fertilizers, explosives, refrigerants, rubber, food, and as a precursor to general-purpose compounds. In particular, ammonia is essential for building a future hydrogen-based society. Meanwhile, there is growing recognition that liquid ammonia is a competitive and viable technology for large-scale and long-distance hydrogen storage and transportation. This is because converting hydrogen into ammonia can allow more hydrogen to be stored and transported over longer distances, and ammonia has a higher hydrogen storage density than liquefied hydrogen, and unlike natural gas-derived hydrogen, ammonia can produce hydrogen in an eco-friendly manner by producing only hydrogen and nitrogen when decomposed. Therefore, the potential market value of eco-friendly ammonia production technology is expected to increase in the future eco-friendly hydrogen-based society, and it is necessary to develop a more eco-friendly method of mass production to utilize ammonia as a renewable energy storage material.

Most conventional ammonia production processes utilize the Haber-Bosch method, which reacts nitrogen and hydrogen in the gas phase. However, ammonia production using the Haber-Bosch process requires high temperature and pressure (approximately 500° C., 200 atm) reaction conditions, requires 40 MJ of energy to produce 1 kg of ammonia, and produces more than 3.45 kg of carbon dioxide. Because one nitrogen atom and three hydrogen atoms are required to produce ammonia, and the strong bonds between the two nitrogen atoms must be broken, ammonia production using the Haber-Bosch method consumes about 1% of the world's energy. The Haber-Bosch process having the high reaction temperature and pressure not only requires a lot of energy, but also has large restrictions in terms of the use of expensive catalysts. Therefore, there is a need to develop technologies that can produce ammonia, an essential compound for agriculture and energy field, at lower temperatures and in an environmentally friendly manner.

Other prior arts related to ammonia production include a method for producing ammonia from nitrogen using electrochemical methods (Z. Geng, et al, Adv. Mater., 30, 2018; M. Nazemi, et al, Nano Energy, 49, 2018; R. D. Milton, et al, Angew. Chem. Int. Ed., 56, 2017), and a method for synthesizing ammonia from nitrogen using photocatalysis (N. Zhang et al, 2018).

Eco-friendly ammonia production technologies include “Electrochemical Ammonia Synthesis Method Using Recycling Process” (Korean Patent No. 10-2186440), which describes a method for electrochemically synthesizing ammonia using recirculation of ammonia production gas; “Ammonia Synthesizer” (Korean Patent No. 10-1870228), which describes a device for electrochemically synthesizing ammonia using an alkaline aqueous solution or molten solution as an electrolyte; and “Apparatus and Method for Producing Ammonia” (Korean Patent No. 10-1541278), which describes a method for producing ammonia using plasma.

The biological process of nitrogen fixation occurs in a wide variety of organisms, and nitrogen-fixing microorganisms in particular have a nitrogen-fixing reaction pathway that converts airborne nitrogen to ammonia via nitrogenase. This pathway allows nitrogen-fixing microorganisms to convert nitrogen to ammonia at room temperature and atmospheric pressure. Studies have been reported on nitrogen-fixing reactions using Azotobacter vinelandii, a representative nitrogen-fixing microorganism, to convert airborne nitrogen to ammonia. Nitrogenase in a nitrogen-fixing microorganism consists of two components, component II, which is the electron donor, and component I, which is the catalytic portion. Component II transfers electrons generated by ATP oxidation to component I, where ammonia is produced by using nitrogen from the air and hydrogen ions from water. However, the electron production and transfer of component II is very slow in microorganisms, and the rate of nitrogen reduction reaction is also very slow due to the large difference in the rate of binding and separation of component I and component II. In addition, due to the limited expression concentration of nitrogenase in wild-type nitrogen-fixing microorganisms, the rate of ammonia production per enzyme is very slow, so it is not suitable for mass production of ammonia using only nitrogen-fixing microorganisms.

As a technique for producing ammonia by modifying nitrogen-fixing microorganisms, the prior art discloses producing ammonia in vitro using a nanoparticle-nitrogenase complex (K. A. Brown et al., Science, 352:6284, 2016). In this article, nitrogenase was extracted from the nitrogen-fixing bacterium Azotobacter vinelandii and combined with CdS nanoparticles to produce ammonia. However, nitrogenases are very sensitive to oxygen and cannot be extracted and used in large quantities because the extraction process is carried out in a strict nitrogen atmosphere, so there are limitations in utilizing them for mass production of ammonia.

Meanwhile, the prior art discloses a conventional technique of producing ammonia in vivo using nanoparticle-microorganism complex (Y. Ding et al., J. Am. Chem. Soc. 141:26 2019). In the article, Azotobacter vinelandii grown in early-mid log phase was exposed to quantum dots such as CdS@ZnS, CdSe@ZnS, InP@ZnS or Cu₂ZnSnS₄@ZnS together to produce ammonia under light-irradiated conditions, but improvement in ammonia production is needed.

Accordingly, the inventors of the present invention conducted research on an environmentally friendly ammonia production method and found that when nitrogen-fixing microorganisms grown in log or exponential phase are cultured in a medium containing selenium zinc shell quantum dots capped with indium phosphide-based core/mercaptopropionic acid, the content of inorganic nanoparticles inside the microorganisms can be increased, and the prepared inorganic nanoparticle-microorganism complex can allow to produce ammonia at a low temperature (30° C.) in an eco-friendly way with excellent efficiency.

### SUMMARY OF THE INVENTION

The object of the present invention is to provide a method for producing ammonia in an environmentally friendly manner using inorganic nanoparticle-microorganism complex.

To accomplish this objective, the present invention provides a method of producing ammonia, comprising:
(a) generating inorganic nanoparticle-microorganism complex by endogenously expressing nitrogenase in a medium containing inorganic nanoparticle quantum dots to which hydrophilic ligands are introduced or by culturing microorganisms to which nitrogenase is exogenously introduced;
(b) irradiating the inorganic nanoparticle-microorganism complex to produce ammonia; and
(c) recovering the generated ammonia.

### BRIEF DESCRIPTION OF DRAWINGS

FIG. 1 shows a schematic illustration of the inorganic nanoparticle-microorganism complex with quantum dots embedded inside Azotobacter vinelandii, a schematic illustration of the quantum dot structure with indium phosphide core/zinc selenide shell as one embodiment of the quantum dots, a schematic illustration of band energy levels of the quantum dots, absorption spectra of indium phosphide core quantum dots and quantum dots with zinc selenide shell capped with indium phosphide core/mercaptopropionic acid, and transmission electron micrograph of quantum dots with zinc selenide shell capped with indium phosphide core/mercaptopropionic acid.

FIG. 2 shows the results of comparing the ammonia production capacity of indium phosphide core/zinc selenide shell after the surface thereof is modified with MPA, GSH, cell penetrating peptide (CPP), galactose, or fructose ligands.

FIG. 3 shows the intracellular quantum dot content depending on incubation time when Azotobacter vinelandii was cultured in a medium containing quantum dots with zinc selenide shell capped with indium phosphide core/mercaptopropionic acid, expressed as percentages and number of quantum dots per cell mass.

FIG. 4 shows the results of ammonia production in response to light irradiation to complexes to which Azotobacter vinelandii without treating quantum dots, Azotobacter vinelandii cultured in a medium containing quantum dots having zinc selenide shell capped with indium phosphide core/mercaptopropionic acid, and Azotobacter vinelandii in which culture is ended are exposed to quantum dots, respectively.

FIG. 5 shows the results of the ammonia production under light irradiation conditions of Azotobacter vinelandii without treating quantum dots with different growth states, and Azotobacter vinelandii cultured in a medium containing quantum dots with zinc selenide shell capped with indium phosphide core/mercaptopropionic acid.

FIG. 6 shows scanning transmission electron micrographs and elemental analysis images by energy dispersive X-ray spectroscopy obtained under conditions in which light of Azotobacter vinelandii cultured to log phase was not irradiated.

FIG. 7 shows transmission electron micrographs and elemental analysis images by energy dispersive X-ray spectroscopy of inorganic nanoparticle-microorganism complex obtained by culturing Azotobacter vinelandii to log phase in a medium without quantum dots, adding thereto quantum dots with zinc selenide shell capped with indium phosphide core/mercaptopropionic acid, and reacting same under light irradiated conditions for 3 hours.

FIG. 8 shows scanning transmission electron micrographs and elemental analysis images by energy dispersive X-ray spectroscopy obtained under conditions that light of an inorganic nanoparticle-microorganism complex obtained by culturing Azotobacter vinelandii in log phase to a medium containing zinc selenide shell quantum dots capped with 50 nM of indium phosphide core/mercaptopropionic acid was not irradiated.

FIG. 9 shows scanning transmission electron micrographs and elemental analysis images by energy dispersive X-ray spectroscopy obtained under conditions that light of an inorganic nanoparticle-microorganism complex obtained by culturing Azotobacter vinelandii in log phase to a medium containing zinc selenide shell quantum dots capped with 200 nM of indium phosphide core/mercaptopropionic acid was not irradiated.

FIG. 10 shows the results of ammonia production under conditions that light of an inorganic nanoparticle-microorganism complex obtained by culturing Azotobacter vinelandii in log phase to a medium containing zinc selenide shell quantum dots capped with 2 nM, 10 nM, 20 nM, 50 nM, 100 nM or 200 nM of indium phosphide core/mercaptopropionic acid was irradiated (left), and the results of cytotoxicity test depending on the concentration of quantum dots in Azotobacter vinelandii irradiated with light for 1 hour (right).

### DETAILED DESCRIPTION AND PREFERRED EMBODIMENTS OF THE INVENTION

Unless otherwise defined, all technical and scientific terms used herein have the same meaning as commonly understood by a person skilled in the art. In general, the nomenclature used herein is well known and in common use in the art.

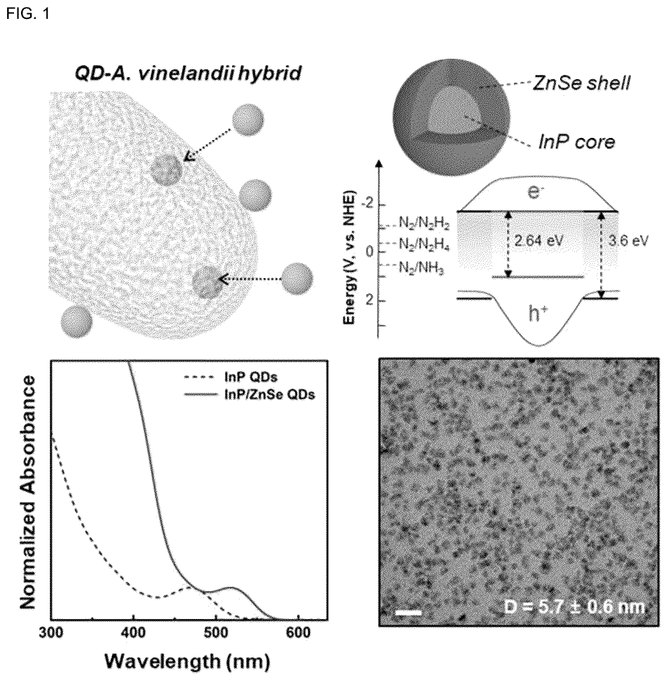

The present invention confirms that efficient ammonia production in an eco-friendly way is possible by using inorganic nanoparticle-microorganism complex with quantum dots embedded inside nitrogen-fixing microorganism. Specifically, the present invention fabricated quantum dots with indium phosphide core/zinc selenide shell and confirmed their band energy level and the energy level of the nitrogen reduction reaction, and found that the nitrogen reduction reaction can be stably induced by electrons generated by the quantum dots to produce ammonia (see FIG. 1).

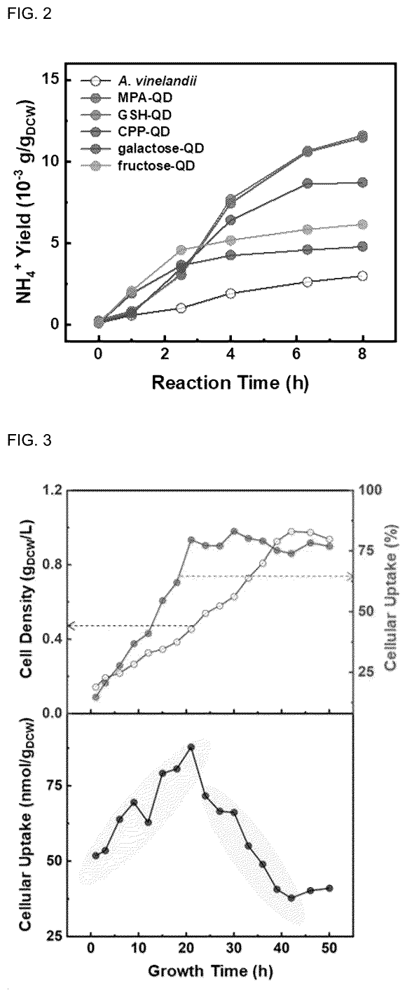

As a result of introducing mercaptopropionic acid (hereinafter, referred to as “MPA”) and L-glutathione (hereinafter, referred to as “GSH”), which modulate the charge on the surface of the quantum dots to increase their incorporation into the microorganism, or attaching cell-penetrating peptide (hereinafter, referred to as “CPP”), or sugar (galactose or fructose) ligands, it was found that the introduction of mercaptopropionic acid or GSH produced the most favorable effect on ammonia production. On the other hand, when MPA was introduced in combination with CPP, the efficiency of ammonia production was found to decrease compared to introducing MPA alone (see FIG. 2).

In the present invention, mercaptopropionic acid is replaced with a hydrophilic ligand for the aqueous dispersion of quantum dots. When the ligand of the quantum dot shell is replaced with mercaptopropionic acid, the thiol group (—SH) of mercaptopropionic acid forms a bond with the surface of the quantum dot, and the carboxyl (—COOH) group provides an affinity for water, resulting in quantum dots with increased dispersion in water. The increased aqueous dispersion of the quantum dots increases their incorporation into the microorganism, which increases the transfer of additional electrons to the nitrogenase, which increases the production of ammonia.

Meanwhile, quantum dots modified with GSH showed a similar level of ammonia production effect as quantum dots modified with MPA, but since GSH contains nitrogen atoms, the experiment was conducted using MPA instead of GSH to utilize only atmospheric nitrogen for ammonia generation.

The extent of incorporation of quantum dots into the microorganism was observed over microorganism growth time, and it was found that the quantum dot incorporation efficiency reached 75% after about 20 hours, and the efficiency was maintained thereafter. However, the amount of incorporated cell per cell dry weight was maximized at about 20 hours (see FIG. 3).

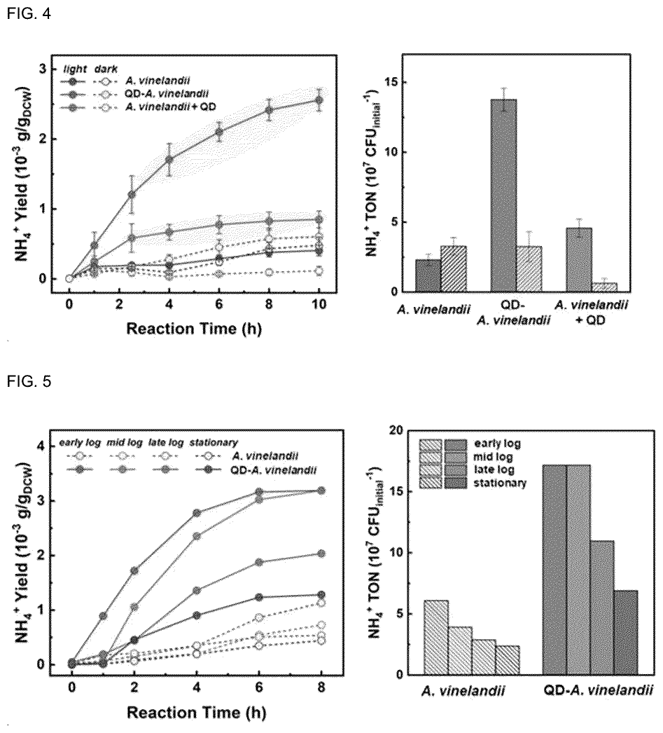

To evaluate the ammonia productivity of microorganism upon introduction of quantum dots, there were provided three experimental groups of Azotobacter vinelandii cultured in Burk's medium for 28 hours, and Azotobacter vinelandii cultured in Burk's medium added with surface-modified quantum dots for 28 hours, Azotobacter vinelandii incubated in Burk's medium for 28 hours and then added with surface-modified quantum dots, and the ammonia productivity was compared depending on whether the light was irradiated and light irradiation time. As a result, Azotobacter vinelandii incubated in Burk's medium added with surface-modified quantum dots for 28 hours and then irradiated with light showed about 4 times more ammonia productivity than the comparison experimental group (see FIG. 4).

Furthermore, to analyze the efficiency of ammonia production depending on the timing of light irradiation, Azotobacter vinelandii was cultured in Burk's medium or Burk's medium supplemented with surface-modified quantum dots at an initial optical density (OD) of 0.005, in early log phase (20 hours), mid log phase (28 hours), late log phase (36 hours), and stationary phase (48 hours), respectively, and then irradiated with light. As a result, it was found that light irradiation in early log phase (20 hours) to mid log phase (28 hours) significantly increased ammonia productivity (see FIG. 5).

It would be apparent to one of ordinary skill in the art that the time in early, mid or late log phase, or stationary phase may be reduced or increased depending on the initial OD value. In one embodiment of the present invention, the phase can be viewed as early log when the OD value is 60-80 times, mid log when the OD value is 80-120 times, late log when the OD value is 120-180 times, and stationary when the OD value is 180-200 times, compared to the initial OD value, but is not limited thereto.

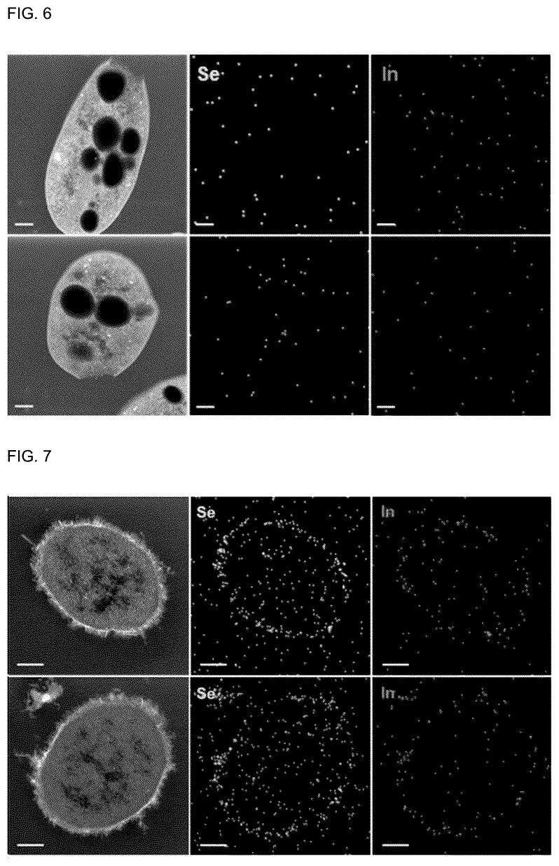

Based on transmission electron micrographs and elemental analysis images by energy dispersive X-ray spectroscopy of inorganic nanoparticle-microorganism complex in Azotobacter vinelandii cultured to log phase, Azotobacter vinelandii cultured to log phase in a medium without quantum dots added with quantum dots with zinc selenide shell capped with indium phosphide core/mercaptopropionic acid, and irradiated for 3 hours, and Azotobacter vinelandii cultured to log phase in a medium containing quantum dots with zinc selenide shell capped with 50 nM or 200 nM of indium phosphide core/mercaptopropionic acid, respectively, it was found that the amount of quantum dots embedded in the microorganism in Azotobacter vinelandii cultured to log phase in a medium containing quantum dots with zinc selenide shell capped with 200 nM of indium phosphide core/mercaptopropionic acid was significantly high (see FIGS. 6 to 9).

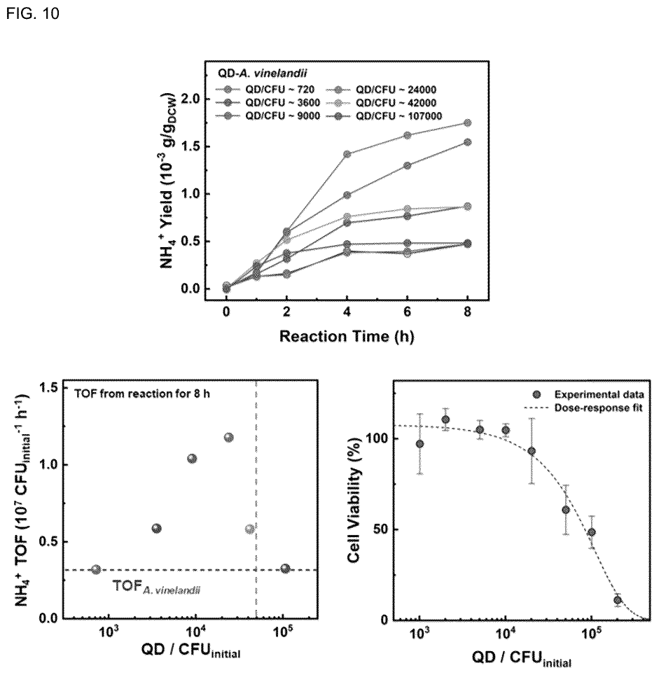

However, the highest ammonia production efficiency was observed in Azotobacter vinelandii cultured to log phase in a medium containing quantum dots with zinc selenide shell capped with 50 nM of indium phosphide core/mercaptopropionic acid, which was attributed to the fact that high concentrations of quantum dots above 100 nM are toxic to microorganisms and inhibit their growth (see FIG. 10).

In 2019, Ding et al. published “Nanorg Microbial Factories: Light-Driven Renewable Biochemical Synthesis Using Quantum Dot-Bacteria Nanobiohybrids,” in which they attempted to produce ammonia using quantum dot-bacteria biohybrids.

However, whereas in the above technology, the increase in ammonia productivity was induced by culturing the microorganism once, recovering it, and then irradiating it with quantum dots and light (see Comparative Example 2), in the present invention, the increase in ammonia productivity was induced by culturing the microorganism in a medium containing quantum dots and further increasing the amount of quantum dots.

In the present invention, zinc selenide shell quantum dots capped with indium phosphide core/mercaptopropionic acid with a quasi-type II core/shell structure were used to improve the electron transfer efficiency of nitrogenase in microorganism.

As a result, a turnover number (TON) of ammonia per cell of up to 1.72×10\hat{ }8 NH₃/cell was obtained in the present invention (see FIG. 5), which is more than four times higher than the result by the method disclosed by Ding et al. above (about 4×10\hat{ }7 NH₃/cell).

Thus, by increasing the content of inorganic nanoparticles in microorganism and effectively transferring electrons generated from inorganic nanoparticles to nitrogenase in microorganism, the present invention can provide an eco-friendly ammonia production method that does not require high temperature and high pressure conditions and does not emit carbon dioxide at a low cost compared to conventional chemical synthesis methods.

Specifically, the present invention relates to a method of producing ammonia comprising:
(a) generating inorganic nanoparticle-microorganism complex by endogenously expressing nitrogenase in a medium containing inorganic nanoparticle quantum dots to which hydrophilic ligands are introduced or by culturing microorganisms to which nitrogenase is exogenously introduced;
(b) irradiating the inorganic nanoparticle-microorganism complex to produce ammonia; and
(c) recovering the generated ammonia.

In the inorganic nanoparticle quantum dot according to the present invention, the core layer can be selected from the group consisting of, but is not limited to, binary compounds such as CdS, CdSe, CdTe, ZnS, ZnSe, ZnTe, AlN, AlP, AlAs, AlSb, GaN, GaP, GaAs, GaSb, InN, InP, InAs, and InSb, and ternary compounds such as CdZnS, CdZnSe. CdZnTe, CdSSe, CdSTe, CdSeTe, ZnSSe, ZnSTe, ZnSeTe, AlGaN, AlGaP, AlGaAs. AlGaSb, AlInN, AlInP, AlInAs, AlInSb, GaInN, GaInP, GaInAs, GaInSb, AlNP, AlNAs, AlNSb, AlPAS, AlPSb, GaNP, GaNAs, GaNSb, GaPAs, GaPSb, InNP, InNAs, InNSb, InPAs, InPSb, CuInS2, CuInSe2, CuGaS2, CuGaSe2, AgInS2, AgInSe2, AgGaS2, and AgGaSe2. The shell layer can be selected from the group consisting of, but is not limited to, binary compounds such as CdS, CdSe, CdTe, ZnS, ZnSe, and ZnTe, and ternary compounds such as CdZnS, CdZnSe. CdZnTe, CdSSe, CdSTe, CdSeTe, ZnSSe, ZnSTe, ZnSeTe.

In the present invention, the quantum dots may be, but are not limited to, quantum dots having an indium phosphide core/zinc selenide shell.

In the present invention, the hydrophilic ligand can be selected from the group consisting of, but is not limited to, mercaptopropionic acid (MPA), L-glutathione (GSH), mercaptoacetic acid, mercaptobutanoic acid, mercaptopentanoic acid, mercaptohexanoic acid, mercaptoheptanoic acid, mercaptooctanoic acid, mercaptononanoic acid, mercaptodecanoic acid, mercaptoundecanoic acid, mercaptododecanoic acid, and L-cysteine.

Preferably, the hydrophilic ligand may be mercaptopropionic acid (MPA) or L-glutathione (GSH).

In the present invention, in step (a), the quantum dots are contained in the medium at a concentration of 20 to 100 nM, preferably 30 to 80 nM, more preferably 40 to 60 nM. This is because if the concentration of quantum dots is less than 20 nM, it is difficult to supply sufficient electrons to the nitrogenase, and if the concentration of quantum dots is greater than 100 nM, it interferes with microbial growth.

In the present invention, the light in step (b) may be characterized in that the microorganism begin to be irradiated at an early log phase to a mid log phase, and the irradiation of the light may be initiated between 14 and 32 hours after incubation of the microorganism, such as between 16 and 30 hours, most preferably between 20 and 28 hours, but the present invention is not limited thereto.

In the present invention, the light may be irradiated in step (b) for more than 2 hours, such as, but not limited to, about 3 hours, about 4 hours, about 5 hours, about 6 hours, about 7 hours, about 8 hours, about 9 hours, about 10 hours, about 15 hours, about 20 hours, about 30 hours, about 40 hours, about 50 hours, about 60 hours, about 70 hours, about 80 hours, about 90 hours, or about 100 hours. In step (b), the light may be irradiated for 2 to 72 hours. That is, the length of time that the light is irradiated in step (b) of the present invention is dependent on the length of time that the microorganism grows and the activity of the nitrogenase therein is maintained, and it would be readily understood by one of ordinary skill in the art that the ammonia production reaction can be induced by irradiating the light as long as the activity of the nitrogenase is maintained.

In the present invention, the microorganism may be selected from the group consisting of, but is not limited to, Clostridium sp., Klebsiella pneumoniae, Paenibacillus polymyxa, Bacillus macerans, Escherichia intermedia, Azotobacter agilis, Azotobacter armeniacus, Azotobacter beijerinckii, Azotobacter chroococcum, Azotobacter nigricans, Azotobacter paspali, Azotobacter salinestris, Azotobacter tropicalis, Azotobacter vinelandii, Rhizibium sp., Achromobacter, Azorhizobium sp., Frankia sp., Pseudomonas sp., Bacillus sp., Nitrobacter sp., Fusarium oxysporum, Cylindrocaropn tonkinese, Bipolaris sorokiniana, and Cyanobacteria sp.

In the present invention, the microorganism may be Azotobacter vinelandii.

In the embodiment of the present invention, the surface of the quantum dots was modified with MPA, but excellent ammonia generation capability was also confirmed for quantum dots modified with GSH.

Hereinafter, the present invention will be described in more detail with reference to examples. These examples are only for illustrating the present invention, and it will be obvious to those skilled in the art that the scope of the present invention should not be construed as being limited by these examples

### Example 1. Synthesis of Quantum Dots and Surface Ligand Exchange Reaction

### 1-1. Synthesis and Purification of Indium Phosphide-Based Core/Shell Quantum Dots

Indium acetate (0.2 mmol), oleic acid (0.6 mmol), n-trioctylphosphine (1 mL) and 1-octadecene (10 mL) were added to a 100 mL three-necked flask, and heated to 120° C. under a low-pressure atmosphere (200 mTorr) for 2 hours. The reactor was then switched to an inert atmosphere and heated to 300° C., and tris-trimethylsilylphosphine (0.1 mmol) prepared in an inert atmosphere was added and reacted for 10 min. Zinc oleate (0.1 mmol) and seleniated n-trioctylphosphine (0.1 mmol) prepared under an inert atmosphere were added to the reactor and heated for 30 min. Zinc oleate (0.2 mmol) and seleniated n-trioctylphosphine (0.2 mmol) prepared under an inert atmosphere were added to the reactor and heated for 30 minutes. After the end of the reaction, the temperature of the reactor was lowered to room temperature to obtain the reaction product. The reaction product (5 mL) was mixed with toluene (5 mL), ethanol (10 mL), and acetone (10 mL), and then precipitated by centrifugation (10000 rpm, 5 min), and the supernatant of the centrifugation product was discarded, and the precipitate was dispersed in toluene (5 mL). The solution of quantum dots dispersed in toluene was mixed with ethanol (10 mL), and acetone (10 mL), and then precipitated via centrifugation (10,000 rpm, 5 min), and the precipitated quantum dots were dispersed in toluene (5 mL). The purification process was repeated twice more, and finally the precipitated quantum dots were dispersed in toluene (5 mL).

As a result, as shown in FIG. 1, the synthesized indium phosphide/zinc selenide core/shell quantum dots exhibited a significant increase in absorbance at a wavelength of 400 nm compared to indium phosphide core quantum dots, and transmission electron microscopy images confirmed that the core/shell quantum dots had a diameter of about 5.7 nm.

### 1-2. Surface Ligand Exchange Reactions of Indium Phosphide-Based Core/Shell Quantum Dots to Mercaptopropionic Acid, L-Cysteine, and Glutathione

Purified quantum dots were dispersed in toluene to make a quantum dot solution (20 uM/1 mL), which was mixed with a ligand exchange reaction solution (mercaptopropionic acid, L-cysteine or glutathione (0.2 M) and tetramethylammonium hydroxide pentahydrate (0.35 M)) dissolved in methanol and stirred for 10 min. Acetone (6 mL) was added to the solution (6 mL) and centrifuged (10,000 rpm, 5 min). The supernatant of the centrifuged product was discarded and the precipitate was dispersed in methanol (5 ml), and then acetone (10 mL) and hexane (10 mL) were added, and the solution was centrifuged (10,000 rpm, 5 min). The centrifuged solid product was dispersed in 5 ml of water and refrigerated. By this ligand exchange reaction, the surface ligands of the quantum dots were exchanged from oleic acid to mercaptopropionic acid, L-cysteine or glutathione.

### 1-3. Method of Preparing Galactose and Fructose Ligands

Lactose or sucrose (4 mmol), cysteamine hydrochloride (4 mmol), borate buffer solution (100 mL, 0.1 M, pH=9) were added to a 500 ml capacity glass bottle and stirred for 5 minutes. To the solution, which became clear, sodium cyanoborohydride (5 g) was added and stirred for another 12 hours. By the reaction, the glucose portion of lactose or sucrose combines with the amine group of cysteamine to form a compound comprising galactose or fructose and a thiol group at both ends. Acetone (20 mL) was added to the 12-hour stirred solution (10 mL) and precipitated by centrifugation (8000 rpm, 10 min). After discarding the supernatant, the precipitated compound was dissolved in water (10 mL), acetone (20 mL) was added, and the compound was precipitated via the centrifugation procedure. The precipitated product was dissolved in water (10 mL) and refrigerated.

### 1-4. Ligand Exchange Reactions of Indium Phosphide-Based Core/Shell Quantum Dots to Galactose and Fructose Compounds or Cell-Permeable Peptides

To the mercaptopropionic acid-capped indium phosphide-based core/shell quantum dot solution (5 mL) dispersed in the water obtained in Example 1-2, a solution of galactose or fructose compound (0.05 M, 5 mL) or cell-permeable peptide solution (0.1 mM, 5 mL) obtained in Example 1-3 was added and sonicated for 10 min. Acetone (10 mL) was added to the mixed solution to precipitate the quantum dots capped with galactose or fructose compound via centrifugation (10000 rpm, 5 min), the supernatant of the centrifugation result was discarded, and the precipitated quantum dots were dispersed in water (5 mL). The quantum dot solution was transferred to a centrifuge tube (Amicon Ultra-15 30K) equipped with a centrifuge filter (molecular weight cut-off=30 kDa), and the quantum dot solution was concentrated to a volume of about 0.5 mL by centrifugation (7,000 rpm, 15 min). Water (4.5 mL) was added to the concentrated quantum dot solution, and the centrifugation filtering process was repeated two more times, and water (4.5 mL) was added to the final concentrated quantum dot solution and refrigerated.

### Example 2. Comparison of Ammonia Generation Effects Depending on Quantum Dot Surface Modification

According to one aspect of the present invention, to prepare the inorganic nanoparticle-microorganism complex, wild-type Azotobacter vinelandii KCTC 2426 was initial cultured in 5 mL of Burk's medium (10 g/L Glucose, 0.64 g/L K₂HPO₄, 0.16 g/L KH₂PO₄, 0.2 g/L NaCl, 0.2 g/L MgSO₄·7H₂O, 0.05 g/L CaSO₄·2H₂O, 0.01 g/L NaMoO₄·2H₂O, 0.003 g/L FeSO₄) for 36 hours at 30° C. After inoculating 1 mL of the initial cell culture into Burk's medium (100 mL), 50 nM of various surface-modified indium phosphide-based core/shell quantum dots prepared in Example 1 were mixed and incubated together in Burk's medium (100 mL) to prepare inorganic nanoparticle-microorganism complex for 30 hours at 30° C. The complex was then irradiated with light for up to 8 hours, and the effects of different ligands on ammonia production were compared.

As a result, as shown in FIG. 2, it was found that indium phosphide-based core/shell quantum dots capped with mercaptopropionic acid and glutathione ligands have productivity of about 11.6 mg/L of final ammonia concentration, which is about four times the productivity of wild-type Azotobacter vinelandii. Furthermore, it was found that productivity decreases in the following order: using cell-permeable peptide, and fructose or galactose-capped quantum dots.

### Example 3. Measurement of Quantum Dot Content Depending on Growth Time of Inorganic Nanoparticle-Microorganism Complex

Wild-type Azotobacter vinelandii KCTC 2426 was initial cultured in 5 mL of Burk's medium (10 g/L Glucose, 0.64 g/L K₂HPO₄, 0.16 g/L KH₂PO₄, 0.2 g/L NaCl, 0.2 g/L MgSO₄·7H₂O, 0.05 g/L CaSO₄·2H₂O, 0.01 g/L NaMoO₄·2H₂O, 0.003 g/L FeSO₄) at 30° ° C. for 36 hours. 1 mL of the initial cell culture with an optical density of 0.5 at a wavelength of 600 nm was inoculated into Burk's medium (100 mL), and then 50 nM of indium phosphide-based core/shell quantum dots capped with mercaptopropionic acid ligands were mixed and incubated together in Burk's medium (100 mL) to prepare inorganic nanoparticle-microorganism complex for 50 hours at 30° C. At approximately 3-hour intervals, 1 mL of the inorganic nanoparticle-microorganism complex culture was taken and centrifuged (12,000 rpm, 1 min), and the precipitated cells were redispersed in PBS solution. After further centrifugation (12,000 rpm, 1 min) and redispersion in PBS solution were repeated three times, 0.38 mL of hydrochloric acid solution (37%) and 0.12 mL of nitric acid solution (70%) were added to the final precipitated inorganic nanoparticle-microorganism complex and the solution was dissolved for 48 hours. To the final dissolved cell solution, 4.5 mL of distilled water was added and the intracellular quantum dot content was measured using inductively coupled plasma spectrometry.

As a result, as shown in FIG. 3, the quantum dot content increased during the first 20 hours of incubation, but after 20 hours, the quantum dot content no longer increased to the level of 75-80%. Meanwhile, the quantum dot content per cell mass increased during the first 20 hours of incubation and decreased after 20 hours.

### Example 4. Comparison of Ammonia Productivity Using Azotobacter vinelandii, Inorganic Nanoparticle-Microorganism Complex, and a Mixture of Azotobacter Vinelandii and Quantum Dots

4-1. Cultivation of Azotobacter vinelandii (Comparative Example 1)

Wild-type Azotobacter vinelandii KCTC 2426 was initial cultured in 5 mL of Burk's medium (10 g/L Glucose, 0.64 g/L K₂HPO₄, 0.16 g/L KH₂PO₄, 0.2 g/L NaCl, 0.2 g/L MgSO₄·7H₂O, 0.05 g/L CaSO₄·2H₂O, 0.01 g/L NaMoO₄·2H₂O, 0.003 g/L FeSO₄) at 30° C. for 36 hours. 1 mL of the initial cell culture was inoculated into Burk's medium (100 mL) and incubated at 30° C. for 20 to 48 hours. After centrifugation (8000 rpm, 5 min) of the culture containing Azotobacter vinelandii, the precipitated cells were redispersed in PBS solution. The process of centrifugation and redispersion in PBS solution was repeated twice, and the amount of PBS solution was adjusted so that the optical density (OD) of the finally precipitated cells was equal to 2 when measured at a wavelength of 600 nm with a spectrophotometer.

4-2. Preparation of a Mixture of Azotobacter vinelandii and Quantum Dots (Comparative Example 2)

When the optical density of Azotobacter vinelandii cells cultured and centrifuged as in Example 4-1 was measured at a wavelength of 600 nm with a spectrophotometer, they were redispersed by adding ½ of the amount of PBS solution adjusted to 2. To 1 mL of the cell solution, 1 mL of a solution of mercaptopropionic acid-capped indium phosphide-based core/shell quantum dots (40 to 4000 nM) dispersed in a PBS solution was mixed so that the optical density of the final cells was 2 when measured at a wavelength of 600 nm and the concentration of the quantum dots was adjusted to 20 to 2000 nM.

### 4-3. Preparation and Cultivation of Inorganic Nanoparticle-Microorganism Complex

In one aspect according to the present invention, to prepare inorganic nanoparticle-microorganism complex, 1 mL of the initial cell culture of Example 4-1 (with an optical density of 0.5 at a wavelength of 600 nm) and indium phosphide-based core/shell quantum dots having several concentrations (2 to 200 nM) were mixed and incubated together in Burk's medium (100 mL) for 20 to 48 hours at 30° C. The optical density at a wavelength of 600 nm of the cell solution at 20, 28, 36, and 48 hours of incubation is 0.3 to 0.4, 0.4 to 0.6, 0.6 to 0.9, and 0.9 to 1, respectively. After centrifugation (8000 rpm, 5 min) of the culture solution containing the indium phosphide-based core/shell quantum dot-microorganism complex, the precipitated cells were redispersed in a PBS solution. The process of centrifugation of the solution and redispersion in PBS solution was repeated twice, and the amount of PBS solution was adjusted to make the optical density (OD) of the precipitated cells to 2 when measured at a wavelength of 600 nm with a spectrophotometer.

4-4. Comparison of Ammonia Productivity Using Azotobacter vinelandii, Inorganic Nanoparticle-Microorganism Complex, and Mixture of Azotobacter vinelandii and Quantum Dots.

After transferring 2 mL of a solution of Azotobacter vinelandii, indium phosphide-based core/shell quantum dot-microorganism complex, or a mixture of Azotobacter vinelandii and quantum dots having an optical density (600 nm) of 2 to a round-bottomed reactor, and then Azotobacter vinelandii, zinc phosphide-based core/shell quantum dot-microbe complex, and mixture of Azotobacter vinelandii and quantum dots were incubated for 8 hours at 30° C. under conditions with or without light irradiation (400 nm wavelength) to the reactor. Depending on the reaction time of 1 h to 8 h, 0.1 mL of the reaction solution was mixed with water (0.9 mL) and centrifuged (12000 rpm, 1 min), and 500 μL of indicator 1 (citric acid (5 wt %)/sodium citrate (5 wt %) and sodium hydroxide (1 M)) was added to 500 μL of the supernatant. To the mixed solution, 250 μL of indicator 2 (0.05 M sodium hypochlorite) and 50 μL of indicator 3 (1 wt % sodium nitroferricyanide) were added and reacted at room temperature for 2 hours. The optical density (655 nm) of the reactant was measured and compared with the optical density of the reactant mixed with a reference solution having known ammonia concentration (0 to 0.2 mM) and an indicator, and then the ammonia production was calculated.

As a result, as shown in FIG. 4, Azotobacter vinelandii, inorganic nanoparticle-microbial complex, and mixture of Azotobacter vinelandii and quantum dots obtained with 28 hours of incubation time exhibited activities per cell mass of 0.41×10\hat{ }−3 g/gDCW, 2.56×10\hat{ }−3 g/gDCW, and 0.85×10\hat{ }−3 g/gDCW, respectively, for a reaction time of 10 hours under light irradiation. In addition, the inorganic nanoparticle-microorganism complex showed similar values to the ammonia productivity of Azotobacter vinelandii under unirradiated conditions. Calculating the turnover number of ammonia production per cell using the concentration of colony forming units in the initial cells after 10 hours of reaction under light irradiation, the inorganic nanoparticle-microorganism complex showed a production of approximately 14×10⁷ NH₃/CFU.

Furthermore, the ammonia production response of Azotobacter vinelandii and the inorganic nanoparticle-microorganism complex was checked by adjusting the cell incubation time to 20, 28, 36 and 48 hours, as described above, and it was found that the ammonia productivity of both Azotobacter vinelandii and the inorganic nanoparticle-microorganism complex decreased when the cell incubation time increased beyond 28 hours, as shown in FIG. 5.

### Example 5. Scanning Transmission Electron Microscopy Sample Preparation and Measurement of Azotobacter vinelandii, Inorganic Nanoparticle-Microorganism Complex, and Mixture of Azotobacter vinelandii and Quantum Dots

### 5-1. Scanning Transmission Electron Microscopy Sample Preparation

As prepared in Example 4-1 to 4-3, 1 mL of Azotobacter vinelandii, inorganic nanoparticle-microorganism complex and mixture of Azotobacter vinelandii and quantum dots with optical density at 600 nm wavelength adjusted to 2 was centrifuged (12000 rpm, 1 min) and redispersed in PBS solution. After the centrifugation and redispersion process was repeated twice, 1 mL of fixative 1 (2.5 wt % glutaraldehyde in 0.1 M phosphate buffer, PB) was added to the final precipitated cells and the chemical fixation process was carried out for 4 hours. The fixed cell solution was centrifuged (12000 rpm, 1 min) and redispersed in 1 mL of PB, and the centrifugation and redispersion in PB process was repeated twice, and fixative 2 (1 wt % osmium tetroxide in 0.1 M PB) was added to the precipitated cells for an additional fixation process of 2 hours. The fixed cell solution was centrifuged (12000 rpm, 1 min) and redispersed in 1 ml of water, and after repeating the centrifugation and redispersion in water process twice, the final precipitated cells were subjected to a dehydration process after adding 1 mL of a mixture of water and ethanol (30 vol % ethanol) for 10 min. The dehydrated cell solution was centrifuged (12,000 rpm, 1 min) and the dehydration and centrifugation process was repeated using 50, 70, 80, 90, and 100 vol % ethanol mixtures. The final centrifuged cells were redispersed in 1 mL of ethanol and the process of centrifugation and redispersion in 1 mL of ethanol was repeated three times. The ethanol-dispersed cell solution was centrifuged (12000 rpm, 1 min), and then 1 mL of propylene oxide was added to the precipitated cells, followed by a 15 min solvent switching process and centrifugation (12000 rpm, 1 min). After the redispersion and centrifugation process was repeated twice in 1 mL of propylene oxide, a mixture of propylene oxide and Spurr's resin (1 mL) mixed in a 3:1 ratio was added to the final precipitated cells and reacted for 30 min. The reaction and centrifugation process was repeated for 30 minutes with adjusting the ratio of propylene oxide and Spurr's resin of 2:1, 1:1, 1:2 and 1:3, and Spurr's resin was finally added to the precipitated cells to undergo a polymer curing process of 12 hours at room temperature and 24 hours at 70° C., and the prepared fixed cells and polymer support were fabricated into section of about 100 nm thickness using an ultramicrotome. The section was placed on a TEM grid (Cu, 200 mesh with carbon support film) to complete the sample preparation.

### 5-2. Scanning Transmission Electron Microscopy Measurement

The scanning transmission electron microscopy sample prepared as in Example 5-1 was loaded into a transmission electron microscopy instrument (FEI, Talos F200×, 200 kV), the measurement mode of the transmission electron microscope was changed to the scanning transmission electron microscopy measurement mode, and the fixed cells supported on the polymer support were explored. After obtaining scanning transmission electron microscopy images of the explored cells, energy dispersive X-ray (EDS) mapping images were obtained by irradiating the cells with an electron beam for 3 min.

FIGS. 6 and 7 show scanning transmission electron microscopy images and elemental analysis of Azotobacter vinelandii and a mixture of Azotobacter vinelandii and quantum dots irradiated with light for 3 hours. In the case of the mixture of Azotobacter vinelandii and quantum dots irradiated with light for 3 hours, no quantum dots were incorporated into the cells, and the quantum dots were adsorbed onto the surface of the cells.

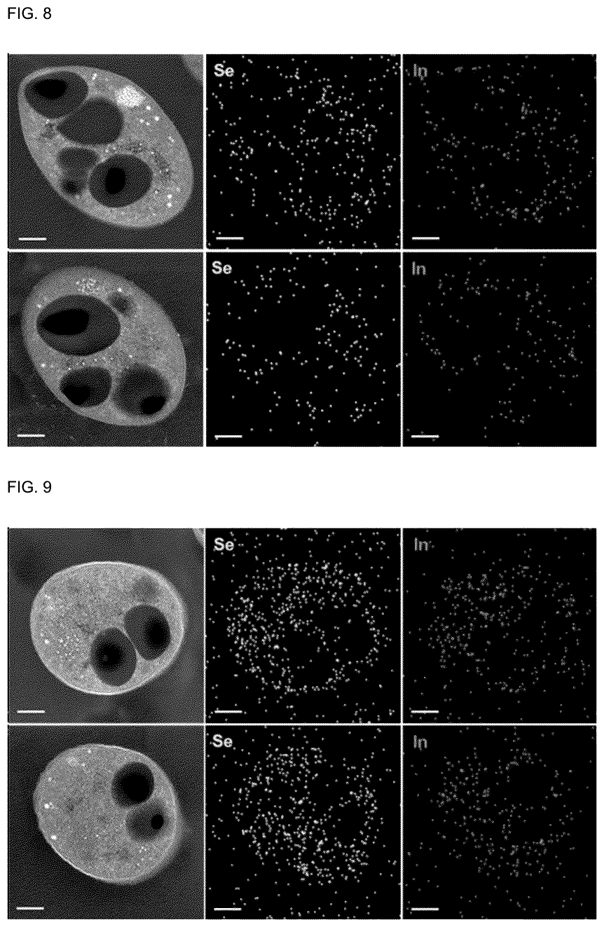

FIGS. 8 and 9 are scanning transmission electron microscopy images and elemental analysis results of inorganic nanoparticle-microorganism complex cultured at quantum dot concentrations of 50 nM and 200 nM in the culture medium. In the case of the inorganic nanoparticle-microorganism complex, it was confirmed that the quantum dots were incorporated into the cells, and the inorganic nanoparticle-microorganism complex cultured in the culture medium containing quantum dots with high concentration showed high quantum dot incorporation.

### Example 6. Evaluation of Cytotoxicity of Inorganic Nanoparticle-Microorganism Complex Under Light Irradiation Condition

Comparing the ammonia productivity of the inorganic nanoparticle-microorganism complex when the concentration of quantum dots in the culture medium was increased to 2 nM to 200 nM as in Example 4, it was found that the ammonia productivity of the inorganic nanoparticle-microorganism complex increased when the concentration of quantum dots in the culture medium was increased to 2 nM to 50 nM, but the productivity decreased when the concentration was increased to 50 nM to 200 nM, as shown in FIG. 10.

Since it was expected to be a result of inhibiting the growth of microorganism when the concentration of quantum dots in microorganism exceeded a certain concentration, resulting in a decrease in ammonia productivity, the cytotoxicity of inorganic nanoparticle-microorganism complex under light irradiation conditions, depending on the concentration of quantum dot treatment.

The inorganic nanoparticle-microorganism complex with an optical density of 2 at 600 nM prepared in Example 4 was diluted with PB by 1.0×10⁷, and 0.1 mL of the diluted cell solution was inoculated into Burk's culture plate (90 mm diameter) by plating method and incubated for 28 hours. The number of colony forming units of the cultured Azotobacter vinelandii and inorganic nanoparticle-microorganism complex was counted and multiplied by 10⁷ to calculate the concentration of cells capable of initial cell differentiation. The cell concentration calculation method was applied to Azotobacter vinelandii and inorganic nanoparticle-microorganism complex irradiated with light for 1 hour to calculate the concentration of cells capable of differentiation after 1 hour of light irradiation. In addition, cell viability was calculated by comparing the concentration of the initially differentiable cell with the concentration of the differentiable cell after 1 hour of light irradiation.

As a result, it was found that the concentration of differentiable cells was halved after 1 hour when the relative number of quantum dots for the inorganic nanoparticle-microorganism complex was about 50,000, as shown in FIG. 10, and it was understood that the cytotoxicity of these quantum dots caused the ammonia productivity to be rather reduced at 50 nM or more.

### INDUSTRIAL APPLICABILITY

The present invention can produce ammonia at lower temperature and pressure conditions compared to the conventional Haber-Bosch method, which produces ammonia under conditions of high temperature and pressure, and can produce ammonia in an environmentally friendly way without the emission of carbon dioxide emitted during the conventional chemical synthesis process, and can be a competitive alternative to the prior art for the production of ammonia, which has unlimited potential as a future energy resource.

Although specific configurations of the present invention have been described in detail, those skilled in the art will appreciate that this description is provided to set forth preferred embodiments for illustrative purposes, and should not be construed as limiting the scope of the present invention. Therefore, the substantial scope of the present invention is defined by the accompanying claims and equivalents thereto.

1. A method of producing ammonia, comprising:
(a) generating inorganic nanoparticle-microorganism complex by endogenously expressing nitrogenase in a medium containing inorganic nanoparticle quantum dots to which hydrophilic ligands are introduced or by culturing microorganisms to which nitrogenase is exogenously introduced;
(b) irradiating the inorganic nanoparticle-microorganism complex to produce ammonia; and
(c) recovering the generated ammonia.

2. The method according to claim 1, wherein the inorganic nanoparticle quantum dots are quantum dots having an indium phosphide core/zinc selenide shell.

3. The method according to claim 1, wherein the hydrophilic ligand may be selected from the group consisting of mercaptopropionic acid (MPA), L-glutathione (GSH), mercaptoacetic acid, mercaptobutanoic acid, mercaptopentanoic acid, mercaptohexanoic acid, mercaptoheptanoic acid, mercaptooctanoic acid, mercaptononanoic acid, mercaptodecanoic acid, mercaptoundecanoic acid, mercaptododecanoic acid, and L-cysteine.

4. The method according to claim 1, wherein in step (a), the quantum dots are contained in the medium at a concentration of 20 to 100 nM.

5. The method according to claim 1, wherein in step (b), the light begins to irradiate the microorganism in an early log phase to a mid log phase.

6. The method according to claim 1, wherein in step (b), the light is irradiated for 2 to 72 hours.

7. The method according to claim 1, wherein the microorganism may be selected from the group consisting of Clostridium sp., Klebsiella pneumoniae, Paenibacillus polymyxa, Bacillus macerans, Escherichia intermedia, Azotobacter agilis, Azotobacter armeniacus, Azotobacter beijerinckii, Azotobacter chroococcum, Azotobacter nigricans, Azotobacter paspali, Azotobacter salinestris, Azotobacter tropicalis, Azotobacter vinelandii, Rhizibium sp., Achromobacter, Azorhizobium sp., Frankia sp., Pseudomonas sp., Bacillus sp., Nitrobacter sp., Fusarium oxysporum, Cylindrocaropn tonkinese, Bipolaris sorokiniana, and Cyanobacteria sp.
